(19) United States
(12) Patent Application Publication
(10) Pub. No.: US 2005/0227363 A1
(43) Pub. Date: Oct. 13, 2005

(54) Methods of monitoring rinsing solutions and related systems and reagents

(76) Inventors: Dong-won Hwang, Gyeonggi-do (KR); Pil-kwon Jun, Gyeonggi-do (KR); Sung-jae Lee, Gyeonggi-do (KR); Jae-seok Lee, Seoul (KR); Sang-mun Chon, Gyeonggi-do (KR); In-hoi Doh, Gyeonggi-do (KR)

Correspondence Address: MYERS BIGEL SIBLEY & SAJOVEC, PO BOX 37428, RALEIGH, NC, 27627, US

(21) Appl. No.: 10/952,510

(22) Filed: Sep. 28, 2004

(30) Foreign Application Priority Data
Apr. 12, 2004 (KR) 2004-24897

(51) Int. Cl. G01N 33/00

(52) U.S. Cl. 436/73; 422/68.1

(57) ABSTRACT

Monitoring metal contamination of a rinsing solution may include providing a sample of the rinsing solution, and mixing the sample of the rinsing solution with a monitoring reagent to provide a monitoring mixture. A property of the monitoring mixture that is dependent on a concentration of a metal in the rinsing solution may then be measured. More particularly, the property of the monitoring mixture may be an absorbency of the monitoring mixture with respect to electromagnetic radiation transmitted through the monitoring mixture. Related systems and reagents are also discussed.

### RELATED APPLICATIONS

The present application claims the benefit of priority from Korean Patent Application Serial No. 2004-24897 filed Apr. 12, 2004, the disclosure of which is hereby incorporated herein in its entirety by reference.

### FIELD OF THE INVENTION

The present invention relates to the filed of microelectronics and more particularly to microelectronics fabrication.

### BACKGROUND OF THE INVENTION

When fabricating integrated circuit devices, a plurality of integrated circuits are fabricated on a single semiconductor wafer. Various processing steps are performed on the wafer to form and pattern various layers included in the integrated circuit devices formed thereon. A semiconductor wafer, for example, may be subjected to processing steps including deposition, diffusion, photolithography, etch, and/or ion implantation in the fabrication of the integrated circuit devices thereon. Once complete, the integrated circuit devices of the wafer may be separated by dicing.

Between processing steps, the semiconductor wafer may be rinsed in a rinsing solution to remove contaminants such as metals, particles, and/or organic materials from the wafer. Rinsing solutions, for example, may include diluted hydrofluoric acid (DHF), SC1 (a cleaning solution developed by RCA Corp. made up of H₂O:H₂O₂:NH₄OH in a typical ratio of approximately 5:1:1), sulfuric acid, and/or ultra de-ionized water. More particularly, one or more semiconductor wafers may be submerged in a bath of the rinsing solution. After a plurality of rinsing operations, a metal contaminant(s) may accumulate in the rinsing solution, and subsequently rinsed wafers may be contaminated by metal(s) accumulated in the rinsing solution.

With a rinsing solution of SC1, a metal contaminant in the rinse solution may be easily transferred to a wafer being rinsed. With a rinsing solution of diluted HF, an unstable metal fluoride may result from a reaction of a metal and the rinse solution, and the metal fluoride may result in oxidation, reduction, and/or pitting on a surface of a wafer being rinsed. Yields of Integrated circuit devices fabricated on a wafer rinsed in a solution contaminated with aluminum (Al) and/or iron (Fe) may be reduced.

### SUMMARY OF THE INVENTION

According to embodiments of the present invention, methods for monitoring metal contamination of a wafer rinsing solution may include providing a sample of the wafer rinsing solution, and mixing the sample of the rinsing solution with a monitoring reagent to provide a monitoring mixture. A property of the monitoring mixture that is dependent on a concentration of a metal in the rinsing solution may then be measured. More particularly, the property of the monitoring mixture may be an absorbency of the monitoring mixture with respect to electromagnetic radiation transmitted through the monitoring mixture.

Providing a sample of the rinsing solution, for example, may include providing a continuous flow of the rinsing solution. In addition, mixing the sample of the rinsing solution with the monitoring reagent may include mixing the continuous flow of the rinsing solution with a continuous flow of the monitoring reagent to provide a continuous flow of the monitoring mixture, and the property of the monitoring mixture may be measured periodically. Moreover, a ratio of the rinsing solution to the monitoring reagent is in the range of approximately 1:1 to approximately 5:1. More particularly, the rinsing solution may be provided at approximately 0.8 milliliters per minute, and the monitoring reagent may be provided at approximately 0.2 milliliters per minute.

In an alternative, providing a sample of the wafer rinsing solution may include providing a discrete sample of the rinsing solution. In addition, mixing the sample of the rinsing solution with the monitoring reagent may include mixing the sample of the rinsing solution with a discrete sample of the monitoring reagent, and discrete samples of the rinsing solution and the monitoring reagent may be mixed periodically. Moreover, a ratio of the discrete sample of the rinsing solution to the discrete sample of the monitoring reagent may be in the range of approximately 1:1 to approximately 5:1. More particularly, the discrete sample of the rinsing solution may be approximately 0.2 milliliters, and the discrete sample of the monitoring reagent may be approximately 0.05 milliliters.

The monitoring reagent may include a chelating agent. More particularly, the chelating agent may include at least one of: an azo type organic compound; an eriochrome cyanine R type organic compound; chromeazurol-S(CAS); C₂₃H₁₃Cl₂Na₃O₉S; an 8-hydroxyquinoline derivative; 8-hydroxyquinoline; 5-sulfo-8-hydroxyquinoline; 5-chloro-7-iodo-8-hydroxyquinoline; 5,7-dichloro-8-hydroxyquinoline; 5.7-dibrome-8-hydroxyquinoline; tiron (1,2-dihydroxy-3,5-benzenedisulfonic acid disodium salt, monohydrate); hydroxy-2-(2-hydroxyphenylazo)benzene; lumogallion (5-chloro-2-hydroxy-3-(2,4-dihydroxyphenylazo)benzenesulfonic acid); and/or pyrocatechol violet (PV pyrocatechol sulfonphthalein).

Moreover, the monitoring reagent may include a chelating agent and a pH controller including at least one of a base and/or an acid. If the wafer rinsing solution is alkaline, the pH controller may be acidic, for example, having a pH in the range of approximately 3 to approximately 11. If the wafer rinsing solution is acidic, the pH controller may be alkaline, for example, having a pH in the range of approximately 4.8 to approximately 7.5. For example, the reagent may include a chelating agent, water, and a pH controller including at least one of a base and/or an acid, and the rinsing solution may include at least one of NH₄OH/H₂O₂/H₂O(SC1), H2SO4/H₂O₂, HCl/H₂O₂/H₂O, HNO₃/HF/H₂O, H₂SO₄/H₂O₂, NH₄OH/H₂O₂/H₂O, HF/H₂O₂, HF/NH₄/H₂O₂, diluted HF, HFNH₄, HF/HNO₃/CH₃COOH, H₃PO₄, HNO₃/H₃PO4/CH₃COOH, and/or de-ionized water.

According to additional embodiments of the present invention, a system for monitoring metal contamination of a wafer rinsing solution in a wafer rinsing bath may include a reservoir, a sampling line, a mixer and an analyzer. The reservoir may be configured to store a monitoring reagent, and the sampling line may be configured to provide a sample of the wafer rinsing solution from the wafer rinsing bath. The mixer may be coupled to the sampling line and to the reservoir, and the mixer may be configured to mix a sample of the rinsing solution with the monitoring reagent to providing a monitoring mixture. The analyzer may be configured to measure a property of the monitoring mixture that is dependent on a concentration of a metal in the rinsing solution. More particularly, the analyzer may be configured to transmit electromagnetic radiation through the monitoring mixture and to measure an absorbency of the monitoring mixture with respect to the electromagnetic radiation transmitted through the monitoring mixture.

For example, the sampling line may be configured to provide a continuous flow of the rinsing solution. In addition, the mixer may be configured to mix the continuous flow of the rinsing solution with a continuous flow of the monitoring reagent to provide a continuous flow of the monitoring mixture, and the analyzer may be configured to periodically measure the property of the continuous flow of the monitoring mixture. Moreover, a ratio of the rinsing solution to the monitoring reagent in the monitoring mixture may be in the range of approximately 1:1 to approximately 5:1. More particularly, the continuous flow of the rinsing solution may be provided at approximately 0.8 milliliters per minute, and the continuous flow of the monitoring reagent may be provided at approximately 0.2 milliliters per minute.

In an alternative, the sample of the wafer rinsing solution may be provided as a discrete sample of the rinsing solution, the discrete sample of the rinsing solution may be mixed with a discrete sample of the monitoring reagent. Moreover, a ratio of the discrete sample of the rinsing solution to the discrete sample of the monitoring reagent in the monitoring mixture may be in the range of approximately 1:1 to approximately 5:1. More particularly, the discrete sample of the rinsing solution may be approximately 0.2 milliliters of the rinsing solution and the discrete sample of the monitoring reagent may be approximately 0.05 milliliters of the monitoring reagent. In addition, discrete samples of the rinsing solution and the monitoring reagent may be mixed periodically.

The monitoring reagent may include a chelating agent. More particularly, the chelating agent may include at least one of an azo type organic compound; an eriochrome cyanine R type organic compound, chromeazurol-S(CAS); C₂₃H₁₃Cl₂Na₃O₉S; an 8-hydroxyquinoline derivative; 8-hydroxyquinoline; 5-sulfo-8-hydroxyquinoline; 5-chloro-7-iodo-8-hydroxyquinoline; 5,7-dichloro-8-hydroxyquinoline; 5.7-dibrome-8-hydroxyquinoline; tiron (1,2-dihydroxy-3,5-benzenedisulfonic acid disodium salt, monohydrate); hydroxy-2-(2-hydroxyphenylazo)benzene; lumogallion (5-chloro-2-hydroxy-3-(2,4-dihydroxyphenylazo)benzenesulfonic acid); and/or pyrocatechol violet (PV pyrocatechol sulfonphthalein).

Moreover, the monitoring reagent comprises a chelating agent and a pH controller including at least one of a base and/or an acid. If the wafer rinsing solution is alkaline, the pH controller may include an acid such as acetic acid. If the wafer rinsing solution is acidic, the pH controller may include a base such ammonium. Moreover, the pH controller selected and the concentration thereof may be determined to provide that a pH of the resulting monitoring mixture is in the range of approximately 3 to approximately 11, and more particularly, in the range of approximately 4.8 to approximately 7.5. Moreover, the monitoring reagent may include a chelating agent, water, and a pH controller including at least one of a base and/or an acid, and the rinsing solution may be selected from at least one of NH₄OH/H₂O₂/H₂O(SC1), H2SO₄/H₂O₂, HCl/H₂O₂/H₂O, HNO₃/HF/H₂O, H₂SO₄/H₂O₂, NH₄OH/H₂O₂/H₂O, HF/H₂O₂, HF/H₂O₂, diluted HF, HFNH₄, HF/HNO₃/CH₃COOH, H₃PO₄, HNO₃/H₃PO4/CH₃COOH, and/or de-ionized water.

According to yet additional embodiments of the present invention, a monitoring reagent for monitoring a level of metal contamination in a rinsing solution may include a chelating agent and a pH controller. The chelating agent forms a complex compound with a metal when mixed with the rinsing solution including the metal, and the pH controller comprising may be an acid or a base. More particularly, he chelating agent may include at least one of an azo type organic compound; an eriochrome cyanine R type organic compound; chromeazurol-S(CAS); C₂₃H₁₃Cl₂Na₃O₉S; an 8-hydroxyquinoline derivative; 8-hydroxyquinoline; 5-sulfo-8-hydroxyquinoline; 5-chloro-7-iodo-8-hydroxyquinoline; 5,7-dichloro-8-hydroxyquinoline; 5.7-dibrome-8-hydroxyquinoline; tiron (1,2-dihydroxy-3,5-benzenedisulfonic acid disodium salt, monohydrate); hydroxy-2-(2-hydroxyphenylazo)benzene; lumogallion (5-chloro-2-hydroxy-3-(2,4-dihydroxyphenylazo)benzenesulfonic acid); and/or pyrocatechol violet (PV pyrocatechol sulfonphthalein).

Moreover, the chelating agent may include at least two of the above referenced compounds. For example, the chelating agent may include an azo type organic compound and at least one of an eriochrome cyanine R type organic compound, chromeazurol-S(CAS), C₂₃H₁₃Cl₂Na₃O₉S, an 8-hydroxyquinoline derivative, 8-hydroxyquinoline, 5-sulfo-8-hydroxyquinoline, 5-chloro-7-iodo-8-hydroxyquinoline, 5,7-dichloro-8-hydroxyquinoline, 5.7-dibrome-8-hydroxyquinoline, tiron (1,2-dihydroxy-3,5-benzenedisulfonic acid disodium salt, monohydrate), hydroxy-2-(2-hydroxyphenylazo)benzene, lumogallion (5-chloro-2-hydroxy-3-(2,4-dihydroxyphenylazo)benzenesulfonic acid), and/or pyrocatechol violet (PV pyrocatechol sulfonphthalein).

If the rinsing solution is alkaline, the pH controller may include an acid such as acetic acid so that the monitoring reagent has a pH level in the range of approximately 1 to 3.0. If the rinsing solution is acidic, the pH controller may include a base such as ammonia so that the monitoring reagent has a pH in the range of approximately 8 to 10. Moreover, the monitoring reagent may include water, the chelating agent may be provided in the range of approximately 0.0001 to approximately 0.1 weight percent of the monitoring reagent, and the pH controller may be provided in the range of approximately 1 to approximately 30 weight percent of the monitoring reagent.

### BRIEF DESCRIPTION OF THE DRAWINGS

FIGS. 1-10 are chemical formulas of chelating agents for use in monitoring reagents according to embodiments of the present invention.

FIG. 11 is a diagram illustrating systems for monitoring contamination of rinsing solutions according to embodiments of the present invention.

FIGS. 12 and 16-19 are graphs illustrating monitoring of contamination according to embodiments of the present invention.

FIGS. 13-15 are graphs illustrating absorbencies of monitoring mixtures as a function of wavelength according to embodiments of the present invention.

FIG. 20 is a graph illustrating absorbencies of a monitoring mixture as a function of nickel contamination of a rinsing solution according to embodiments of the present invention.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. As used herein, the term and/or includes any and all combinations of one or more of the associated listed items.

It will be understood that although the terms first and second are used herein to describe various reagents, mixtures, chelating agents, etc., these reagents, mixtures, chelating agents, etc. should not be limited by these terms. These terms are only used to distinguish one reagents, mixtures, chelating agents, etc. from another reagents, mixtures, chelating agents, etc. Thus, a first reagents, mixtures, chelating agents, etc. discussed below could be termed a second reagents, mixtures, chelating agents, etc. and similarly, a second reagents, mixtures, chelating agents, etc. could be termed a first reagents, mixtures, chelating agents, etc. without departing from the teachings of the present invention. Like numbers refer to like elements throughout.

According to embodiments of the present invention, a monitoring reagent can be used to monitor a level of metal contamination in a wafer rinsing solution. More particularly, the monitoring reagent can be mixed with the rinsing solution to provide a monitoring mixture, and a property of the monitoring mixture can be measured wherein the property is dependent on a concentration of a metal in the rinsing solution. Moreover, the monitoring reagent can be mixed with the rinsing solution and the property of the monitoring mixture can be measured in real time.

The monitoring reagent may include an organic compound, and when mixed with a rinsing solution with metal contamination therein, the organic compound may react with ions of the contaminating metal to form a complex compound. The monitoring reagent may also include de-ionized water and a pH controller. Accordingly, a measure of the complex compound formed in the monitoring mixture can be used to determine a degree of metal contamination in the rinse solution. More particularly, an absorbency of electromagnetic radiation (such as light) through the monitoring mixture may be dependent on a concentration of the complex compound formed in the monitoring mixture. A concentration of the complex compound in the monitoring mixture may thus be determined by transmitting electromagnetic radiation through the monitoring mixture and detecting an intensity of the electromagnetic radiation through the monitoring mixture. Particular organic compounds and/or wavelengths of electromagnetic radiation may be selected for detection of particular metal contaminants in the rinsing solution.

The organic compound of the monitoring reagent may be a chelating agent, and the chelating agent may be mixed with de-ionized water and a pH controller. The chelating agent may be in the range of approximately 0.0001 to approximately 0.1 weight percent of the monitoring reagent, and the pH controller may be in the range of approximately 1 to approximately 30 weight percent monitoring reagent. The pH controller may be either acidic or alkaline depending on the rinsing solution being monitored. For example, if the rinsing solution is an alkaline rinsing solution such as SC1, the pH controller may be an acid such as acetic acid. If the rinsing solution is an acidic rinsing solution such as diluted HF (DHF), the pH controller may be a base such as ammonia water. Monitoring reagents according to embodiments of the present invention may thus be used to monitor acidic and/or alkaline rinsing solutions such as NH₄OH/H₂O₂/H₂O(SC1), H2SO4/H₂O₂, HCl₂O₂/H₂O, HNO₃/HF/H₂O, H₂SO₄/H₂O₂, NH₄OH/H₂O₂/H₂O, HF/H₂O₂, HF/NH₄/H₂O₂, diluted HF, HFNH₄, HF/HNO₃/CH₃COOH, H₃PO₄, HNO₃/H₃PO4/CH₃COOH, and/or de-ionized water.

Accordingly, a rinsing solution may be maintained in a rinsing bath, and the same rinsing solution may be used to rinse pluralities of wafers, with a metal contamination of the rinsing solution increasing over time. A sample of the rinsing solution may be mixed with a sample of the monitoring reagent. More particularly, a volume ratio of the sample of the rinsing solution and the sample of the monitoring reagent may be in the range of approximately 1:1 to approximately 5:1, and a pH of the monitoring mixture may be in the range of approximately 4.8 to approximately 7.5. An absorbency of light through the monitoring mixture can thus be periodically measured to provide substantially real time monitoring of metal contamination.

First Monitoring Reagents

According to first embodiments of the present invention, a first monitoring reagent may including a first chelating agent, de-ionized water, and a pH controller, and the first chelating agent may be an azo type organic compound. The pH controller may be provided in an amount of approximately 1 weight percent to approximately 30 weight percent of the monitoring reagent, and the first chelating agent may be provided in an amount of approximately 0.0001 weight percent to approximately 0.1 weight percent of the monitoring reagent. More particularly, the azo type organic compound may be expressed by the chemical formula,
R1-N═N—R2,
where N is nitrogen, where R1 is a naphthalene radical including a pyridyl radical and/or a hydroxyl radical, and where R2 is a benzene radical including a hydroxyl radical and/or a naphthalene radical.

If R1 is a pyridyl radical and R2 is 1,3-dihydroxybenzene, the azo type organic compound can be 4-(2-pyridylazo)resorcinol (PAR). If R1 is a pyridyl radical and R2 1-hydroxynaphthalene, the azo type organic compound can be 1-(2-pyridylazo)-2-naphthol (PAN). In an alternative, a mixture of PAR and PAN can be used as the first chelating agent in the monitoring reagent.

An azo type organic compound may react with ions of group II transitional metals and with ions of heavy metals such as Ni₂+, Cu₂+, Fe₂+, Zn₂+, Pb₂+, and/or Pt₂+. Accordingly, a monitoring reagent including an azo type organic compound may be used to monitor concentrations of group II transition metals and/or heavy metals in a rinsing solution. An absorbency of the monitoring reagent including an azo type organic compound with respect to electromagnetic radiation may be particularly sensitive to reaction with group II transition metals and/or heavy metals at wavelengths of approximately 480 nm.

Moreover, the pH controller may be provided in the first monitoring reagent so that the mixture of the first monitoring reagent and the rinsing solution being monitored has a pH in range of approximately 3 to approximately 11. Accordingly, the type and quantity of the pH controller provided in the first monitoring reagent may vary depending on the rinsing solution being monitored. The first chelating agent may react with group II transitional metals and/or ions of heavy metals when a pH of the mixture of the first monitoring reagent and the rinsing solution is in the range of approximately 4.8 to approximately 7.5.

Second Monitoring Reagents

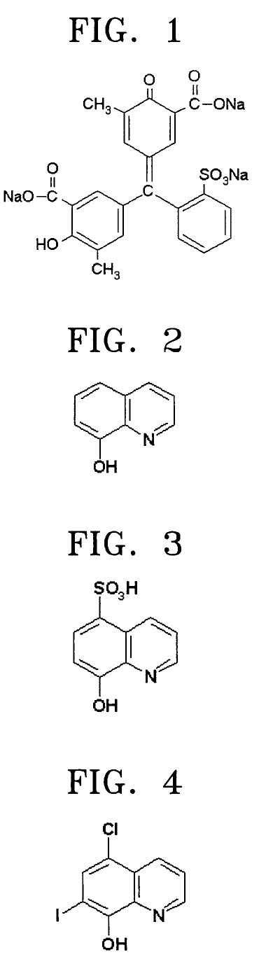

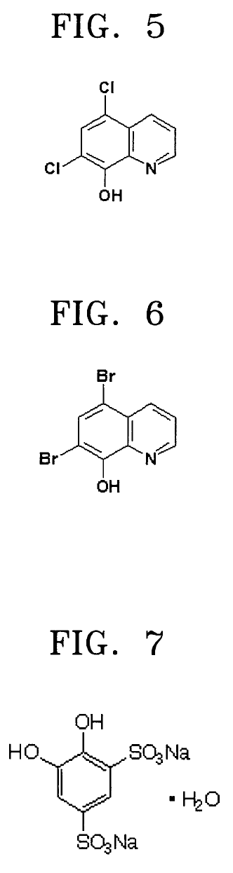

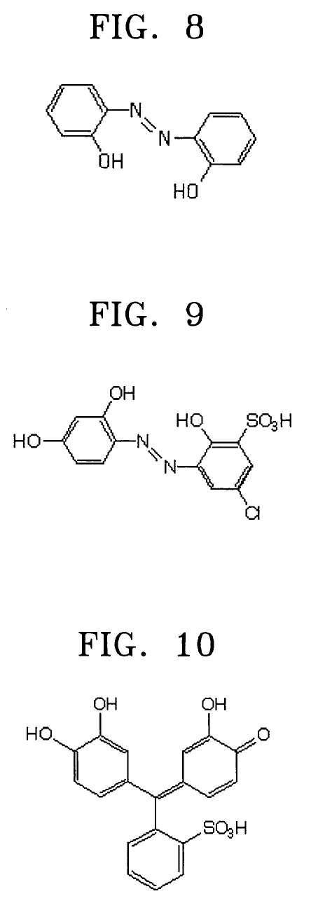

According to second embodiments of the present invention, a second monitoring reagent may include a second chelating agent, de-ionized water, and pH controller. More particularly, the second chelating agent may include an erichrome cyanine R such as expressed by the chemical formula of FIG. 1. In the chemical formula of FIG. 1, CH₃ is a methyl radical, OH is a hydroxyl radical, NaO is an oxy sodium radical, and SO₃Na is a sodium sulfuric radical. In an alternative or in addition, the second chelating agent may include chromeazurol-S(CAS) (C₂₃H₁₃Cl₂Na₃O₉S). In additional alternatives or in addition, the second chelating agent may include an 8-hydroxyquinoline derivative such as: 8-hydroxyquinoline as expressed by the chemical formula of FIG. 2; 5-sulfo-8-hydroxyquinoline as expressed by the chemical formula of FIG. 3; 5-chloro-7-iodo-8-hydroxyquinoline as expressed by the chemical formula of FIG. 4; 5,7-dichloro-8-hydroxyquinoline as expressed by the chemical formula of FIG. 5; and/or 5.7-dibrome-8-hydroxyquinoline as expressed by the chemical formula of FIG. 6. In other alternatives or in addition, the second chelating agent may include: tiron (1,2-dihydroxy-3,5-benzenedisulfonic acid disodium salt, monohydrate) as expressed by the chemical formula of FIG. 7; hydroxy-2-(2-hydroxyphenylazo)benzene as expressed by the chemical formula of FIG. 8; lumogallion (5-chloro-2-hydroxy-3-(2,4-dihydroxyphenylazo)benzenesulfonic acid) as expressed by the chemical formula of FIG. 9; and/or pyrocatechol violet (PV pyrocatechol sulfonphthalein) as expressed by the chemical formula of FIG. 10. The second chelating agent may include one of the above referenced organic compounds discussed with respect to FIGS. 1-10 or combinations thereof.

The pH controller may be provided in an amount of approximately 1 weight percent to approximately 30 weight percent of the monitoring reagent, and the second chelating agent may be provided in an amount of approximately 0.0001 weight percent to approximately 0.1 weight percent of the monitoring reagent. According to the second embodiments of the present invention, second chelating agents may react with ions of group III metals such as aluminum, gallium, and/or indium. Because aluminum contamination may be particularly damaging to integrated circuit devices, monitoring reagents including second chelating agents according to second embodiments of the present invention may be particularly useful in monitoring concentrations of aluminum in a rinsing solution. An absorbency of the second chelating agent with respect to electromagnetic radiation according to the second embodiments of the present invention may be particularly sensitive to reaction with group III metals (such as aluminum) at wavelengths in the range of approximately 520 nm to approximately 530 nm. Accordingly, a concentration of a group III metal in a wafer rinsing solution may be monitored by mixing a monitoring reagent according to second embodiments of the present invention with a sample of the rinsing solution and measuring an absorbency of electromagnetic radiation in the range of approximately 520 μm to approximately 530 nm through the mixture.

Moreover, the pH controller may be provided in the second monitoring reagent so that the mixture of the second monitoring reagent and the rinsing solution being monitored has a pH in the range of approximately 4.8 to approximately 7.5. Accordingly, the type and quantity of the pH controller provided in the second monitoring reagent may vary depending on the rinsing solution being monitored. The second chelating agent may react with group III metals when a pH of the mixture of the second monitoring reagent and the rinsing solution is in the range of approximately 4.8 to approximately 7.5.

Third Monitoring Reagents

According to third embodiments of the present invention, a third monitoring reagent may include a mixture of the first and second chelating agent discussed above, de-ionized water, and a pH controller. The pH controller may be provided in an amount of approximately 1 weight percent to approximately 30 weight percent of the third monitoring reagent. The third chelating agent may be provided such that both of the first and second chelating agents are provided in an amount of approximately 0.0001 weight percent to approximately 0.1 weight percent of the third monitoring reagent. Moreover, the first and second chelating agents may be mixed by a weigh ratio in the range of approximately 1:1 to approximately 1:5 to provide the third chelating agent. Accordingly, the third monitoring reagent (including a mixture of the first and second chelating agents) may be used to monitor concentrations of a group III metal(s), a group II transition metal(s) and/or a heavy metal(s) in a rinsing solution at the same time.

Moreover, the pH controller may be provided in the third monitoring reagent (including both the first and second chelating agents) so that the mixture of the third monitoring reagent and the rinsing solution being monitored has a pH in the range of approximately 4.8 to approximately 7.5. Accordingly, the type and quantity of the pH controller provided in the third monitoring reagent may vary depending on the rinsing solution being monitored.

When monitoring an alkaline rinsing solution such as SC1 having a pH in the range of approximately 7.5 to approximately 10.5, for example, an acidic pH controller such as acetic acid may be added to the third monitoring reagent to provide a pH of the third monitoring reagent in the range of approximately 1 to approximately 3. By mixing the third monitoring reagent having a pH in the range of approximately 1 to approximately 3 with the rinsing solution having a pH in the range of approximately 7.5 to approximately 10.5 in a volume ratio of approximately 1:1 to approximately 1:5, the mixture of the third monitoring reagent and the rinsing solution may be provided with a pH in the range of approximately 4.8 to approximately 7.5.

When monitoring an acidic rinsing solution such as DHF having a pH in the range of approximately 2 to approximately 6, for example, an alkaline pH controller such as ammonia water may be added to the third monitoring reagent to provide a pH of the third monitoring reagent in the range of approximately 8 to approximately 10. By mixing the third monitoring reagent having a pH in the range of approximately 8 to approximately 10 with the rinsing solutiong having a pH in the range of approximately 2 to approximately 6 in a volume ratio of approximately 1:1 to approximately 1:5, the mixture of the third monitoring reagent and the rinsing solution may be provided with a pH in the range of approximately 4.8 to approximately 7.5.

Monitoring Systems

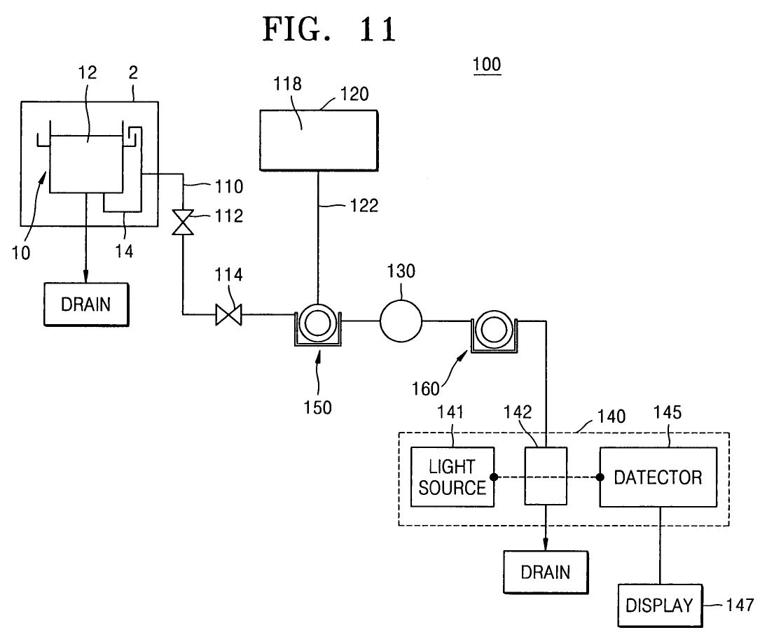

A system 100 for monitoring metal contamination of a wafer rinsing solution 12 in a wafer rinsing bath 10 according to embodiments of the present invention is illustrated in FIG. 11. As shown in FIG. 11, a wet station may include a wafer rinsing bath 10 containing the wafer rinsing solution 12 and a circulation line 14 providing circulation of the wafer rinsing solution 12 in the bath 10. The rinsing solution 12 may include SC1, diluted HF, NH₄OH/H₂O₂/H₂O (SC1), H₂SO₄/H₂O₂, HCl/H₂O₂/H₂O, HNO₃/HF/H₂O, H₂SO₄/H₂O₂, NH₄OH/H₂O₂/H₂O, HF/H₂O₂, HF/NH₄/H₂O₂, diluted HF, HFNH₄, HF/HNO₃/CH₃COOH, H₃PO₄, HNO₃/H₃PO₄/CH₃COOH, and/or ultra de-ionized water.

Semiconductor wafers may be rinsed by immersion in the rinsing solution 12 to remove contaminants therefrom. Contaminants removed from the wafers, however, may accumulate in the rinsing solution 12, and when concentrations of contaminants in the rinsing solution are sufficiently high, damage to subsequently rinsed wafers may result. Accordingly, the rinsing solution 12 should be drained from the bath 10 before undesirable concentrations of contaminants accumulate in the rinsing solution, and fresh rinsing solution may be replaced in the bath 10.

A monitoring reagent 118 according to embodiments of the present invention is provided in a reagent storage bath 120. As discussed above, the monitoring reagent may include a chelating agent, a pH controller, and de-ionized water. More particularly, the chelating agent may include: an azo type organic compound; PAN; PAR; an erichrome cyanine R; chromeazurol-S(CAS) (C₂₃H₁₃Cl₂Na₃O₉S); an 8-hydroxyquinoline derivative; 8-hydroxyquinoline; 5-sulfo-8-hydroxyquinoline; 5-chloro-7-iodo-8-hydroxyquinoline; 5,7-dichloro-8-hydroxyquinoline; 5.7-dibrome-8-hydroxyquinoline; tiron (1,2-dihydroxy-3,5-benzenedisulfonic acid disodium salt, monohydrate); hydroxy-2-(2-hydroxyphenylazo)benzene; lumogallion (5-chloro-2-hydroxy-3-(2,4-dihydroxyphenylazo)benzenesulfonic acid); and/or pyrocatechol violet (PV pyrocatechol sulfonphthalein); and/or combinations thereof. If the rinsing solution 12 is alkaline, the pH controller of the monitoring reagent may be acidic. If the rinsing solution 12 is acidic, the pH controller of the monitoring reagent may be alkaline.

A sampling line 110 may be coupled to the circulation line 14 to provide samples of the rinsing solution 12, and a sampling line 122 may be coupled to the reagent storage bath 120 to provide samples of the monitoring reagent 118. Samples of the rinsing solution 12 and the monitoring reagent 118 may be mixed using mixer 130 to provide a mixture of the rinsing solution 12 and the monitoring reagent 118. The mixture of the rinsing solution and monitoring reagent can then be monitored using measurement system 140 to measure a property of the monitoring mixture that is dependent on a concentration of a contaminant (such as a metal) in the rinsing solution 12.

The measuring system 140 may measure an absorbency of electromagnetic radiation (such as light) through the mixture of the rinsing solution and monitoring reagent provided from the mixer 130. More particularly, the measuring system 140 may include a light source 141, a light detector 145, and a cell 142 (such as a quartz UV cell) through which the mixture flows. Accordingly, an absorbency of light through the mixture flowing through the cell 142 may be monitored, and the absorbency of light through the mixture may be dependent on a concentration of a metal contaminant(s) in the rinsing solution 12. As discussed above, a chelating agent in the monitoring reagent 118 may react with a metal(s) contaminant in the rinsing solution 12 to create a complex compound, and an absorbency of light through the mixture may be dependent on a concentration of the resulting complex compound in the mixture. An increase in metal contamination in the rinsing solution may result in an increase in the resulting complex compound in the mixture and an increase in absorbency of light in the mixture. An increase in absorbency of light through the mixture above a predetermined threshold may thus be used to indicate that an allowable level of metal contamination in the rinsing solution 12 has been exceeded.

The wavelength of light generated by the light source 141 may be selected based on the metal contaminant(s) being monitored in the rinsing solution 12 and/or the chelating agent(s) used in the monitoring reagent 118. Moreover, multiple wavelengths of light may be used to monitor multiple metal contaminants. The detector 145, for example, may include an ultraviolet ray spectrum photometric scale detector, and/or a variable wavelength type ultraviolet ray/visible ray detector. A variable wavelength typed ultraviolet ray/visible ray detector may be used to detect absorbencies of light at multiple wavelengths to thereby monitor multiple metal contaminants in the rinsing solution 12 at the same time.

As shown in FIG. 1, the monitoring reagent sampling line 122 and the rinsing solution sampling line 110 may be coupled to a first pump 150, such as a peristaltic pump that that is capable of continuously transferring a uniform volumes of the monitoring reagent and the rinsing solution to the mixer 130 by revolving a roller over an elastic tube. For example, separate tubes could be provided for the monitoring reagent and the rinsing solution in a same pump 150 with a common roller. Moreover, valves 112 and 114 (such as needle valves) may be provided to control a flow of the rinsing solution 12 though the sampling line 110. In an alternative, separate pumps could be provided for the reagent sampling line 122 and the solution sampling line 110. While a peristaltic pump is discussed above, other pumps could be used to control flows of the monitoring reagent and the rinsing solution. In yet other alternatives, flow through sampling lines 110 and/or 122 may be induced and/or controlled through other means such as a gravity feed and/or valves.

As further shown in FIG. 11, a second pump 160 may provide a controlled flow of the mixture from the mixer 130 to the monitoring apparatus 140, and the second pump 160 may also be a peristaltic pump. In alternatives, flow from the mixer 130 to the monitoring apparatus 140 may be induced and/or controlled through other means such as a gravity feed and/or valves.

Moreover, samples of the rinsing solution 12 and the monitoring reagent 118 may be taken continuously or discretely. For example, a continuous flow of the rinsing solution 12 may be provided through the sampling line 110 at a rate of approximately 3.2 ml/minute, and a continuous flow of the monitoring reagent 118 may be provided through the sampling line 122 at a rate of approximately 0.8 ml/minute. The separate flows of monitoring reagent and rinsing solution may be combined in mixer 130, and a constant flow of the mixture may be provided to the monitoring apparatus. Accordingly the monitoring apparatus may provide a continuous output of absorbency measurements overtime.

In an alternative, discrete samples of the rinsing solution and the monitoring reagent may be provided to the mixer 130. For example, each sample may include 0.8 ml of rinsing solution and 0.2 ml of monitoring reagent, and samples may be taken four times a minute. Accordingly, samples may be mixed and measured every fifteen seconds, and the output may provide discrete measurements at intervals of approximately fifteen seconds.

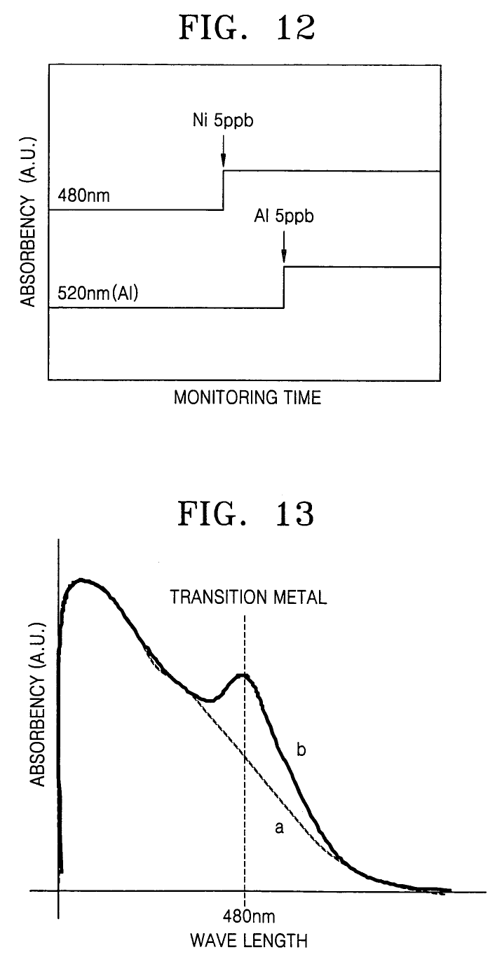

An output from the detector 145 may be provided on a display 147 such as a monitor, and the output may be provided in a format such as that illustrated in FIG. 12. As shown in FIG. 12, a variation in light absorbency (Y-axis) may be provided as a function of time (X-axis). Moreover, variations of light absorption at different wavelengths may be detected and displayed to monitor different contaminants. For example, light absorbency through the mixture of the rinsing solution and monitoring reagent may be monitored at approximately 480 nm and in the range of approximately 520 mm to approximately 530 nm to monitor nickel and aluminum contamination of the rinsing solution.

Operations of monitoring contamination of the rinsing solution 12 of FIG. 11 are discussed below. A sample of the rinsing solution 12 from the bath 10 is provided to the mixer 130, and the sample of the rinsing solution 12 is mixed with monitoring reagent 118 from the bath 120 at mixer 130 to provide a monitoring mixture. More particularly, a flow of the rinsing solution 12 and a flow of the monitoring reagent 118 into the mixer 130 may be controlled using pump 150 such as a peristaltic pump. More particularly, the flows of the rinsing solution 12 and the monitoring reagent 118 may be controlled so that a volume ratio of the rinsing solution 12 to the monitoring reagent 118 in the monitoring mixture thereof is in the range of approximately 1:1 to 5:1. Moreover, the pH controller of the monitoring reagent 118 may be provided such that a pH of the resulting monitoring mixture is in the range of approximately 4.8 to approximately 7.5.

The monitoring mixture of monitoring reagent and rinsing solution from the mixer 130 may then be provided through the cell 142 of the monitoring apparatus 140, and an absorbency of the monitoring mixture may be measured using light source 141 and detector 145. Moreover, a flow of the monitoring mixture through the cell 142 may be controlled using pump 160, and the absorbency of the monitoring mixture may be measured at a wavelength of approximately 480 nm, at a wavelength of approximately 520 nm to approximately 530 nm, or at wavelengths of approximately 480 nm and approximately 520 nm to approximately 530 nm. For example, a wavelength of approximately 480 nm may be used to monitor contamination of a transition metal such as nickel, and a wavelength of approximately 520 nm to approximately 530 nm may be used to monitor contamination of a metal such as aluminum.

Continuous flows of the rinsing solution and the monitoring reagent into the mixer 130 and a continuous flow of the monitoring mixture from the mixer 130 through the cell 142 may provide real time monitoring. In an alternative, discrete samples of the rinsing solution and monitoring reagent may be mixed and measured to provide periodic contamination monitoring. Accordingly, contamination of the rinsing solution 12 in the bath 10 can be monitored in real time, and contamination measurements can be provided between and/or during different wafer rinse operations. As shown in FIG. 12, an introduction of nickel contamination into the rinsing solution 12 may be indicated by an increase in light absorbency of the monitoring mixture at a wavelength of approximately 480 nm. An introduction of Aluminum contamination into the rinsing solution 12 may be indicated by an increase in light absorbency of the monitoring mixture at a wavelength of approximately 520 nm to approximately 530 nm. More particularly, nickel and aluminum contamination can be monitored using the third monitoring reagent discussed above including a mixture of the first and second chelating agents.

### EXAMPLES

The following Examples illustrate monitoring reagents according to embodiments of the present invention. The Examples, however, are not exhaustive and the embodiments of the present invention are not limited by the Examples.

### Example 1

In a first example, a monitoring reagent including a mixture of first and second chelating agents is used to monitor nickel contamination in an SC1 rinsing solution. More particularly, the monitoring reagent of the first example includes 0.002 weight percent of 4-(2-pyridylazo)resorcinol(PAR) (a first chelating agent), 0.006 weight percent of eriochrome cyanine R (a second chelating agent), 12 weight percent of acetic acid (a pH controller), and de-ionized water mixed to provide an aqueous solution having a pH of 2.17.

The rinsing solution of SC1 having a pH of 10 is mixed with the monitoring reagent at a ratio of 4:1. The dotted line graph of FIG. 13 (labeled “a”) illustrates absorbencies of light through the monitoring mixture of SC1 (without metal contamination) and the monitoring reagent as a function of the wavelength of light.

By way of comparison, the rinsing solution of SC1 having a pH of 10 is contaminated with 10 parts-per-billion of nickel, and the contaminated SC1 rinsing solution is mixed with the monitoring reagent at a ratio of 4:1. The solid line graph of FIG. 13 (labeled “b”) illustrates the absorbencies of light through the monitoring mixture of contaminated SC1 and the monitoring reagent as a function of the wavelength of light. The nickel thus reacts with the PAR and/or the eriochrome cyanine R to create a complex nickel compound, and a resulting difference in absorbency is most apparent at approximately 480 nm.

Accordingly, the monitoring reagent of Example 1 can be used to monitor nickel contamination in SC1 by measuring an absorbency of the monitoring mixture using light having a wavelength of approximately 480 nm.

### Example 2

In a second example, a monitoring reagent including a mixture of first and second chelating agents is used to monitor copper contamination in an SC1 rinsing solution. More particularly, the monitoring reagent of the second example includes 0.002 weight percent of 4-(2-pyridylazo)resorcinol(PAR) (a first chelating agent), 0.006 weight percent of eriochrome cyanine R (a second chelating agent), 12 weight percent of acetic acid (a pH controller), and de-ionized water mixed to provide an aqueous solution having a pH of 2.17.

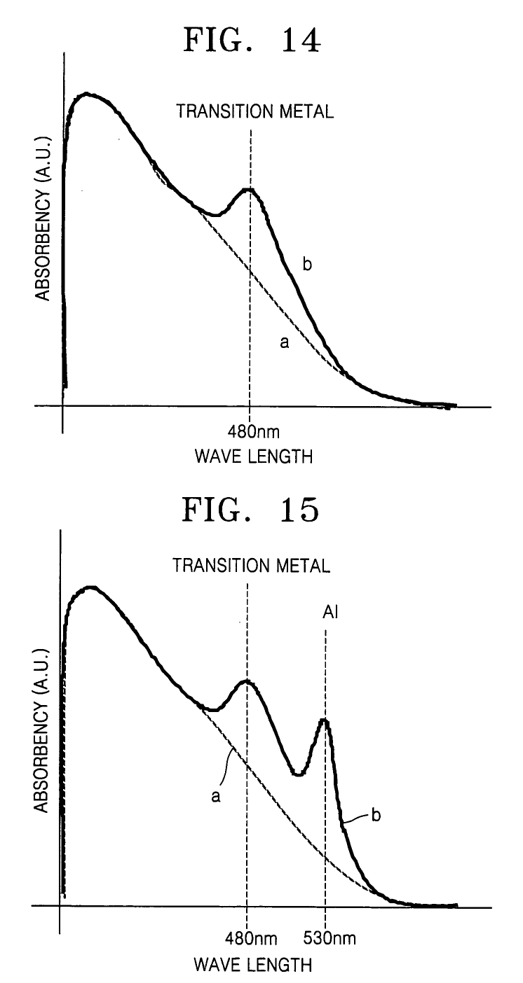

The rinsing solution of SC1 having a pH of 10 is mixed with the monitoring reagent at a ratio of 4:1. The dotted line graph of FIG. 14 (labeled “a”) illustrates absorbencies of light through the monitoring mixture of SC1 (without metal contamination) and the monitoring reagent as a function of the wavelength of light.

By way of comparison, the rinsing solution of SC1 having a pH of 10 is contaminated with 10 parts-per-billion of copper, and the contaminated SC I rinsing solution is mixed with the monitoring reagent at a ratio of 4:1. The solid line graph of FIG. 14 (labeled “b”) illustrates absorbencies of light through the monitoring mixture of contaminated SC1 and the monitoring reagent as a function of the wavelength of light. The copper thus reacts with the PAR and/or the eriochrome cyanine R to create a complex copper compound, and a resulting difference in absorbency is most apparent at approximately 480 nm.

Accordingly, the monitoring reagent of Example 2 can be used to monitor copper contamination in SC1 by measuring an absorbency of the monitoring mixture using light having a wavelength of approximately 480 nm.

### Example 3

In a third example, a monitoring reagent including a mixture of first and second chelating agents is used to monitor nickel and aluminum contamination in an SC1 rinsing solution. More particularly, the monitoring reagent of the third example includes 0.002 weight percent of 4-(2 pyridylazo)resorcinol (a first chelating agent), 0.006 weight percent of eriochrome cyanine R (a second chelating agent), 12 weight percent of acetic acid (a pH controller), and de-ionized water mixed to provide an aqueous solution having a pH of 2.17.

The rinsing solution of SC1 having a pH of 10 may be mixed with the monitoring reagent at a ratio of 4:1. The dotted line graph of FIG. 15 (labeled “a”) illustrates absorbencies of light through the monitoring mixture of SC1 (without metal contamination) and the monitoring reagent as a function of the wavelength of light.

By way of comparison, the rinsing solution of SC1 having a pH of 10 is contaminated with 10 parts-per-billion of nickel and with 10 parts-per-billion of aluminum, and the contaminated SC1 rinsing solution is mixed with the monitoring reagent at a ratio of 4:1. The solid line graph of FIG. 15 (labeled “b”) illustrates absorbencies of light through the monitoring mixture of contaminated SC1 and the monitoring reagent as a function of the wavelength of light. The nickel and aluminum thus react with the PAR and/or the eriochrome cyanine R to create a complex nickel and aluminum compounds, and a resulting difference in absorbency is most apparent at approximately 480 nm and at approximately 520 nm to 530 nm.

Accordingly, the monitoring reagent of Example 3 can be used to monitor nickel and aluminum contamination in SC1 by measuring an absorbency of the monitoring mixture using light having a wavelength of approximately 480 nm and approximately 520 nm to 530 nm. More particularly, the monitoring reagent of Example 3 can be used to monitor nickel contamination in SC1 by measuring an absorbency of the monitoring mixture using light having a wavelength of approximately 480 nm, and the monitoring reagent of Example 3 can be used to monitor aluminum contamination in SC1 by measuring an absorbency of the monitoring mixture using light having a wavelength of approximately 520 nm to approximately 530 nm. Both contaminants can be monitored at the same time by providing that both wavelengths of light are generated by light source 141 and measured at detector 145.

### Example 4

In a fourth example, a monitoring reagent including a mixture of first and second chelating agents is used to monitor nickel and aluminum contamination in an SC1 rinsing solution. As discussed above, a monitoring reagent including a mixture of first and second chelating agents can be used to monitor nickel and aluminum contamination in an SC1 rinsing solution (having a pH of 10). Moreover, nickel and aluminum contamination in the SC1 rinsing solution can be separately detectable. The monitoring reagent of the fourth example includes 0.002 weight percent of 4-(2-pyridylazo)resorcinol (a first chelating agent), 0.006 weight percent of eriochrome cyanine R (a second chelating agent), 12 weight percent of acetic acid (a pH controller), and de-ionized water mixed to provide an aqueous solution having a pH of 2.17.

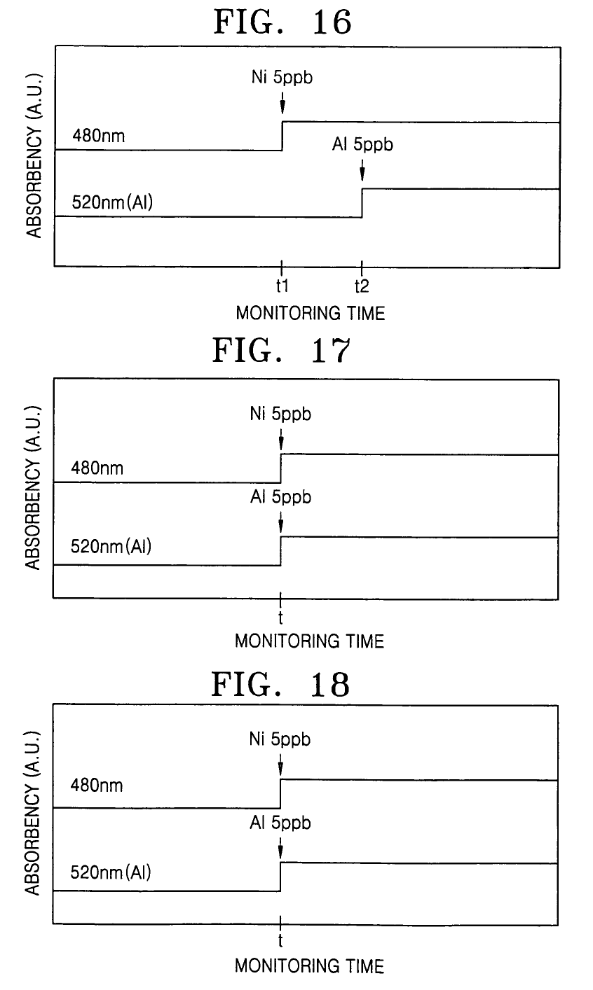

Continuous flows and/or discrete samples of the SC1 rinsing solution and the monitoring reagent are mixed at a ratio of 4:1 using the apparatus of FIG. 11 during a wafer rinsing operation to provide a monitoring mixture. Moreover, a flow of the monitoring mixture is provided through cell 142 to monitor an absorbency of the monitoring mixture with respect to light having wavelengths of approximately 480 nm and approximately 520 nm. The display 147 provides the absorbency information illustrated in FIG. 16 based on output from the detector 145. As shown in FIG. 16, the absorbency of the monitoring mixture through the cell 142 is monitored substantially in real time with respect to light at wavelengths of approximately 480 nm and approximately 520 nm.

With reference to FIGS. 11 and 16, nickel is introduced into the SC1 rinsing solution 12 in the bath 10 at time t₁ to provide a concentration of nickel in the SC1 rinsing solution of 5 parts-per-billion. At approximately time t₁, the nickel contaminated rinsing solution is sampled through the sampling line 110, mixed with the monitoring reagent 118 in mixer 130, and the monitoring mixture of nickel contaminated rinsing solution and monitoring reagent is monitored using light source 141 and detector 145. Reaction of the nickel with the 4-(2-pyridylazo)resorcinol (PAR) increases an absorbency of the monitoring mixture with respect to light have a wavelength of approximately 480 nm. Accordingly, the information of FIG. 16 provided on the display 147 indicates an increase in light absorbency of the monitoring mixture at approximately 480 nm signaling the nickel contamination. While there may be some delay between the introduction of the nickel contamination into the rinsing solution 12 in the bath 10, until the increase in absorbency is indicated on display 147, a continuous flow and/or discrete samples of the rinsing solution and the monitoring reagent to the mixer can provide substantially real time monitoring.

Similarly, aluminum can be introduced into the SC1 rinsing solution 12 in the bath 10 at time t₂ to provide a concentration of aluminum in the SC1 rinsing solution of 5 parts-per-billion. At approximately time t₂, the aluminum contaminated rinsing solution is sampled through the sampling line 110, the sampled rinsing solution is mixed with the monitoring reagent 118 in mixer 130, and the monitoring mixture of nickel contaminated rinsing solution and monitoring reagent is monitored using light source 141 and detector 145. Reaction of the aluminum with the eriochrome cyanine R of the monitoring reagent increases an absorbency of the monitoring mixture with respect to light having a wavelength of approximately 520 nm. Accordingly, the information of FIG. 16 provided on the display 147 indicates an increase in light absorbency of the monitoring mixture at approximately 520 nm signaling the aluminum contamination. While there may be some delay between the introduction of the aluminum contamination into the rinsing solution 12 in the bath 10, until the increase in absorbency is indicated on display 147, a continuous flow and/or discrete samples of the rinsing solution and the monitoring reagent to the mixer can provide substantially real time monitoring.

As further shown in FIG. 16, the introduction of nickel into the rinsing solution does not significantly affect the absorbency of the monitoring mixture with respect to light having a wavelength of approximately 520 nm. Similarly, the introduction of aluminum into the rinsing solution does not significantly affect the absorbency of the monitoring mixture with respect to light having a wavelength of approximately 480 nm. Accordingly, the monitoring reagent with two different chelating agents can be used to separately monitor different contaminants in the rinsing solution in substantially real time.

### Example 5

In a fifth example, a monitoring reagent including a mixture of first and second chelating agents is used to monitor nickel and aluminum contamination in an SC1 rinsing solution. Moreover, nickel and aluminum contamination in the SC1 rinsing solution are separately detectable. The monitoring reagent of the fifth example includes 0.001 weight percent of 4-(2-pyridylazo)resorcinol (a first chelating agent), 0.0001 weight percent of eriochrome cyanine R (a second chelating agent), 5 weight percent of acetic acid (a pH controller), and de-ionized water mixed to provide an aqueous solution.

Continuous flows and/or discrete samples of the SC1 rinsing solution and the monitoring reagent are mixed at a ratio of 1:1 using the apparatus of FIG. 11 during a wafer rinsing operation to provide a monitoring mixture. Moreover, a flow of the monitoring mixture is provided through cell 142 to monitor an absorbency of the monitoring mixture with respect to light having wavelengths of approximately 480 nm and approximately 520 nm. The display 147 provides the absorbency information illustrated in FIG. 17 based on output from the detector 145. As shown in FIG. 17, the absorbency of the monitoring mixture through the cell 142 can be monitored substantially in real time with respect to light at wavelengths of approximately 480 nm and approximately 520 nm.

With reference to FIGS. 11 and 17, nickel and aluminum are introduced into the SC1 rinsing solution 12 in the bath 10 at time t to provide a concentration of nickel in the SC1 rinsing solution of 5 parts-per-billion and a concentration of aluminum in the SC1 rinsing solution of 5 parts-per-billion. At approximately time t, the nickel and aluminum contaminated rinsing solution is sampled through the sampling line 110, mixed with the monitoring reagent 118 in mixer 130, and the monitoring mixture of nickel and aluminum contaminated rinsing solution and monitoring reagent is monitored using light source 141 and detector 145. Reaction of the nickel with the 4-(2-pyridylazo)resorcinol (PAR) increases an absorbency of the monitoring mixture with respect to light having a wavelength of approximately 480 nm, and reaction of aluminum with the eriochrome cyanine R increases an absorbency of the monitoring mixture with respect to light having a wavelength of approximately 520 nm.

Accordingly, the information of FIG. 17 provided on the display 147 indicates an increase in light absorbency of the monitoring mixture at approximately 480 nm and at approximately 520 nm signaling the nickel and aluminum contamination. While there may be some delay between the introduction of the nickel and aluminum contamination into the rinsing solution 12 in the bath 10, until the increase in absorbency is indicated on display 147, a continuous flow and/or discrete samples of the rinsing solution and the monitoring reagent to the mixer can provide substantially real time monitoring. When aluminum and nickel are introduced into the rinsing solution 12 at approximately the same time t, the absorbency of the monitoring mixture increases with respect to light having wavelengths of approximately 480m and approximately 520 nm at approximately the same time t.

### Example 6

In a sixth example, a monitoring reagent including a mixture of first and second chelating agents is used to monitor nickel and aluminum contamination in an SC1 rinsing solution. Moreover, nickel and aluminum contamination in the SC1 rinsing solution can be separately detectable. The monitoring reagent of the sixth example includes 0.1 weight percent of 4-(2-pyridylazo)resorcinol (a first chelating agent), 0.1 weight percent of eriochrome cyanine R (a second chelating agent), 10 weight percent of acetic acid (a pH controller), and de-ionized water mixed to provide an aqueous solution.

Continuous flows and/or discrete samples of the SC1 rinsing solution and the monitoring reagent are mixed at a ratio of 3:1 using the apparatus of FIG. 11 during a wafer rinsing operation to provide a monitoring mixture. Moreover, a flow of the monitoring mixture is provided through cell 142 to monitor an absorbency of the monitoring mixture with respect to light having wavelengths of approximately 480 nm and approximately 520 nm. The display 147 provides the absorbency information illustrated in FIG. 18 based on output from the detector 145. As shown in FIG. 18, the absorbency of the monitoring mixture through the cell 142 can be monitored substantially in real time with respect to light at wavelengths of approximately 480 nm and approximately 520 nm.

With reference to FIGS. 11 and 18, nickel and aluminum are introduced into the SC1 rinsing solution 12 in the bath 10 at approximately time t to provide a concentration of nickel in the SC1 rinsing solution of 5 parts-per-billion and a concentration of aluminum in the SC1 rinsing solution of 5 parts-per-billion. At approximately time t, the nickel and aluminum contaminated rinsing solution is sampled through the sampling line 110, mixed with the monitoring reagent 118 in mixer 130, and the monitoring mixture of nickel and aluminum contaminated rinsing solution and monitoring reagent is monitored using light source 141 and detector 145. Reaction of the nickel with the 4-(2-pyridylazo)resorcinol (PAR) increases an absorbency of the monitoring mixture with respect to light having a wavelength of approximately 480 nm, and reaction of aluminum with the eriochrome cyanine R increases an absorbency of the monitoring mixture with respect to light having a wavelength of approximately 520 nm.

Accordingly, the information of FIG. 18 provided on the display 147 indicates an increase in light absorbency of the monitoring mixture at approximately 480 nm and at approximately 520 nm signaling the nickel and aluminum contamination. While there may be some delay between the introduction of the nickel and aluminum contamination to the rinsing solution 12 in the bath 10, until the increase in absorbency is indicated on display 147, a continuous flow and/or discrete samples of the rinsing solution and the monitoring reagent to the mixer may provide substantially real time monitoring. When aluminum and nickel are introduced into the rinsing solution 12 at approximately the same time t, the absorbency of the monitoring mixture increases with respect to light having wavelengths of approximately 480 nm and approximately 520 nm at approximately the same time t.

### Example 7

In a seventh example, a monitoring reagent including a mixture of first and second chelating agents is used to monitor nickel and aluminum contamination in an SC1 rinsing solution. Moreover, nickel and aluminum contamination in the SC1 rinsing solution can be separately detectable. The monitoring reagent of the seventh example includes 0.001 weight percent of 4-(2-pyridylazo)resorcinol (a first chelating agent), 0.002 weight percent of eriochrome cyanine R (a second chelating agent), 5 weight percent of acetic acid (a pH controller), and de-ionized water mixed to provide an aqueous solution.

Continuous flows and/or discrete samples of the SC1 rinsing solution and the monitoring reagent are mixed at a ratio of 1:1 using the apparatus of FIG. 11 during a wafer rinsing operation to provide a monitoring mixture. Moreover, a flow of the monitoring mixture is provided through cell 142 to monitor an absorbency of the monitoring mixture with respect to light having wavelengths of approximately 480 nm and approximately 520 nm. The display 147 provides the absorbency information illustrated in FIG. 19 based on output from the detector 145. As shown in FIG. 19, the absorbency of the monitoring mixture through the cell 142 can be monitored substantially in real time with respect to light at wavelengths of approximately 480 nm and approximately 520 nm.

With reference to FIGS. 11 and 19, nickel and aluminum are introduced into the SC1 rinsing solution 12 in the bath 10 at approximately time t to provide a concentration of nickel in the SC1 rinsing solution of 5 parts-per-billion and a concentration of aluminum in the SC1 rinsing solution of 5 parts-per-billion. At approximately time t, the nickel and aluminum contaminated rinsing solution is sampled through the sampling line 110, mixed with the monitoring reagent 118 in mixer 130, and the monitoring mixture of nickel and aluminum contaminated rinsing solution and monitoring reagent is monitored using light source 141 and detector 145. Reaction of the nickel with the 4-(2-pyridylazo)resorcinol (PAR) increases an absorbency of the monitoring mixture with respect to light having a wavelength of approximately 480 nm, and reaction of aluminum with the eriochrome cyanine R increases an absorbency of the monitoring mixture with respect to light having a wavelength of approximately 520 nm.

Accordingly, the information of FIG. 19 provided on the display 147 indicates an increase in light absorbency of the monitoring mixture at wavelengths of approximately 480 nm and at approximately 520 nm signaling the nickel and aluminum contamination. While there may be some delay between the introduction of the nickel and aluminum contamination into the rinsing solution 12 in the bath 10, until the increase in absorbency is indicated on display 147, a continuous flow and/or discrete samples of the rinsing solution and the monitoring reagent to the mixer can provide substantially real time monitoring. When aluminum and nickel are introduced into the rinsing solution 12 at approximately the same time t, the absorbency of the monitoring mixture increases with respect to light having wavelengths of approximately 480 nm and approximately 520 nm at approximately the same time t.

### Example 8

In an eighth example, a monitoring reagent including a mixture of first and second chelating agents is used to monitor nickel contamination in an SC1 rinsing solution. The monitoring reagent of the eighth example includes 0.002 weight percent of 4-(2-pyridylazo)resorcinol (a first chelating agent), 0.006 weight percent of eriochrome cyanine R (a second chelating agent), 5 weight percent of acetic acid (a pH controller), and de-Ionized water mixed to provide an aqueous solution.

Continuous flows and/or discrete samples of the SC1 rinsing solution and the monitoring reagent are mixed at a ratio of 4:1 using the apparatus of FIG. 11 to provide a monitoring mixture. Moreover, a flow of the monitoring mixture is provided through cell 142 to monitor an absorbency of the monitoring mixture with respect to light having wavelengths of approximately 480 nm and approximately 520 nm.

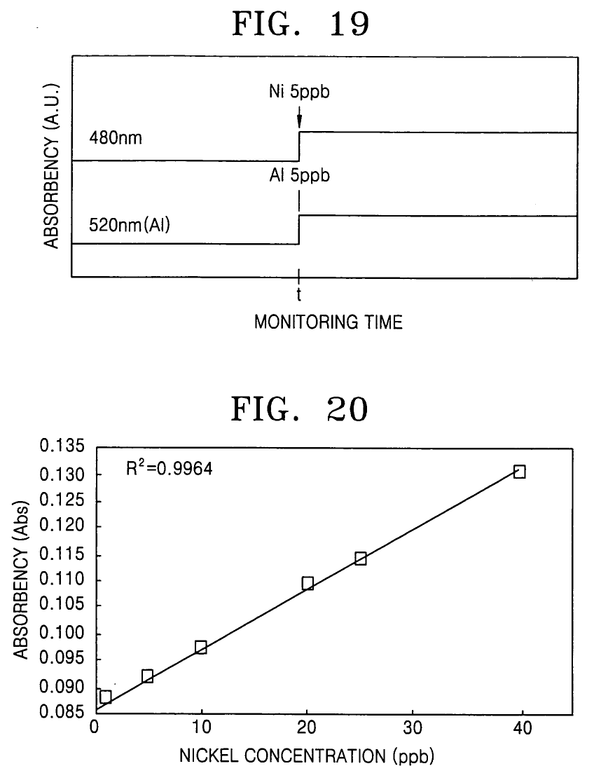

With reference to FIGS. 11 and 20, different concentrations of nickel are introduced into the SC1 rinsing solution 12 in the bath 10. Moreover, absorbencies of the monitoring mixture with respect to light having a wavelength of approximately 480 nm are measured to determine a relationship between concentrations of nickel in the rinsing solution and absorbencies of the resulting monitoring mixture. With reference to FIG. 20, a substantially linear relationship between the concentration of nickel in the SC1 rinsing solution and absorbencies of the resulting monitoring mixture exists with respect to light having a wavelength of approximately 480 nm.

More particularly, a concentration of nickel in the SC1 rinsing solution is increased incrementally with the absorbency of the resulting monitoring mixture being measured for each incremental increase of the nickel concentration. The measured absorbencies are plotted on a graph with respect to the known nickel concentrations as shown in FIG. 20, and linear regression is used to determine a linear function of absorbency with respect to nickel concentration. As shown in FIG. 20, the resulting linear function has a correlation coefficient (R) of approximately 0.9982 (i.e. R².0.9964). Accordingly, monitoring reagents according to embodiments of the present invention can be used to provide relatively precise measurements of contaminants in a rinsing solution in substantially real time.

A monitoring apparatus as discussed above with respect to FIG. 11, for example, may provide a graph illustrating a concentration of contamination and/or absorbencies as a function of time using display 147. A graphic display, however, is not required. For example, the display 147 may provide only an indication that either contaminants in the rinsing solution are within acceptable thresholds or that contaminants in the rinsing solution exceed acceptable thresholds. More particularly, the measuring system 140 may measure absorbencies of light through the monitoring mixture at one or more wavelengths, and a warning signal may be generated when a measured absorbency exceeds a predetermined threshold. In response to the warning signal, the display 147 may provide a warning that the rinsing solution is contaminated and should be replaced before further use. In addition or in an alternative, another warning (such as an audible warning) may be provided when contamination of the rinsing solution exceeds a predetermined threshold.

While the present invention has been particularly shown and described with reference to embodiments thereof, it will be understood by those skilled in the art that various changes in form and details may be made therein without departing from the spirit and scope of the invention as defined by the appended claims and their equivalents.

1. A method for monitoring metal contamination of a rinsing solution, the method comprising:
providing a sample of the rinsing solution;
mixing the sample of the rinsing solution with a monitoring reagent to provide a monitoring mixture; and
measuring a property of the monitoring mixture that is dependent on a concentration of a metal in the rinsing solution.

2. A method according to claim 1 wherein measuring a property of the monitoring mixture comprises measuring an absorbency of the monitor mixture with respect to electromagnetic radiation transmitted through the monitoring mixture.

3. A method according to claim 1 wherein providing a sample of the rinsing solution comprises providing a continuous flow of the rinsing solution.

4. A method according to claim 3 wherein mixing the sample of the rinsing solution with the monitoring reagent comprises mixing the continuous flow of the rinsing solution with a continuous flow of the monitoring reagent to provide a continuous flow of the monitoring mixture.

5. A method according to claim 4 wherein measuring the property of the monitoring mixture comprises periodically measuring the property of the continuous flow of the monitoring mixture.

6. A method according to claim 4 wherein a volume ratio of the rinsing solution to the monitoring reagent in the monitoring mixture is in the range of approximately 1:1 to approximately 5:1.

7. A method according to claim 4 wherein providing the continuous flow of the rinsing solution comprises providing approximately 0.8 milliliters of the rinsing solution per minute and wherein providing the continuous flow of the monitoring reagent comprises providing approximately 0.2 milliliters per minute of the monitoring reagent.

8. A method according to claim 1 wherein providing a sample of the rinsing solution comprises providing a discrete sample of the rinsing solution, wherein mixing the sample of the rinsing solution with a monitoring reagent comprises mixing the sample of the rinsing solution with a discrete sample of the monitoring reagent.

9. A method according to claim 8 wherein the discrete sample of the rinsing solution comprises approximately 0.2 milliliters and the discrete sample of the monitoring reagent comprises approximately 0.05 milliliters.

10. A method according to claim 8 wherein a volume ratio of the discrete sample of the rinsing solution to the discrete sample of the monitoring reagent is in the range of approximately 1:1 to approximately 5:1.

11. A method according to claim 8 wherein discrete samples of the rinsing solution and the monitoring reagent are mixed periodically.

12. A method according to claim 1 wherein the reagent comprises a chelating agent.

13. A method according to claim 12 wherein the chelating agent comprises at least one of an azo type organic compound, an eriochrome cyanine R type organic compound, chromeazurol-S(CAS), C₂₃H₁₃Cl₂Na₃O₉S, an 8-hydroxyquinoline derivative, 8-hydroxyquinoline, 5-sulfo-8-hydroxyquinoline, 5-chloro-7-iodo-8-hydroxyquinoline, 5,7-dichloro-8-hydroxyquinoline, 5.7-dibrome-8-hydroxyquinoline, tiron (1,2-dihydroxy-3,5-benzenedisulfonic acid disodium salt, monohydrate), hydroxy-2-(2-hydroxyphenylazo)benzene, lumogallion (5-chloro-2-hydroxy-3-(2,4-dihydroxyphenylazo)benzenesulfonic acid), and/or pyrocatechol violet (PV pyrocatechol sulfonphthalein).

14. A method according to claim 1 wherein the monitoring reagent comprises a chelating agent and a pH controller including at least one of a base and/or an acid.

15. A method according to claim 14 wherein the rinsing solution is alkaline and wherein the pH controller is acidic.

16. A method according to claim 14 wherein the rinsing solution is acidic and wherein the pH controller is alkaline.

17. A method according to claim 14 wherein the monitoring mixture has a pH level in the range of approximately 3 to 11.

18. A method according to claim 17 wherein the monitoring mixture has a pH level in the range of approximately 4.8 to 7.5.

19. A method according to claim 1 wherein the reagent comprises a chelating agent, water, and a pH controller including at least one of a base and/or an acid.

20. A method according to claim 1 wherein the rinsing solution comprises at least one of NH₄OH/H₂O₂/H₂O(SC1), H₂SO₄/H₂O₂, HCl/H₂O₂/H₂O, HNO₃/HF/H₂O, HF/H₂O₂, HF/NH₄F/H₂O, diluted HF, HF/NH₄F, HF/HNO₃/CH₃COOH, H₃PO₄, HNO₃/H₃PO₄/CH₃COOH, and/or de-ionized water.

21. A system for monitoring metal contamination of a rinsing solution in a rinsing bath, the system comprising:
a reservoir configured to store a monitoring reagent;
a sampling line configured to provide a sample of the rinsing solution from the rinsing bath;
a mixer coupled to the sampling line and to the reservoir, the mixer being configured to mix a sample of the rinsing solution with the monitoring reagent to providing a monitoring mixture; and
an analyzer configured to measure a property of the monitoring mixture that is dependent on a concentration of a metal in the rinsing solution.

22. A system according to claim 21 wherein the analyzer is configured to transmit electromagnetic radiation through the monitoring mixture and to measure an absorbency of the monitoring mixture with respect to the electromagnetic radiation transmitted through the monitoring mixture.

23. A system according to claim 21 wherein the sampling line is configured to provide a continuous flow of the rinsing solution.

24. A system according to claim 23 wherein the mixer is configured to mix the continuous flow of the rinsing solution with a continuous flow of the monitoring reagent to provide a continuous flow of the monitoring mixture.

25. A system according to claim 24 wherein the analyzer is configured to periodically measure the property of the continuous flow of the monitoring mixture.

26. A system according to claim 24 wherein a volume ratio of the rinsing solution to the monitoring reagent in the monitoring mixture is in the range of approximately 1:1 to approximately 5:1.

27. A system according to claim 24 wherein providing the continuous flow of the rinsing solution comprises providing approximately 0.8 milliliters of the rinsing solution per minute and wherein providing the continuous flow of the monitoring reagent comprises providing approximately 0.2 milliliters per minute.

28. A system according to claim 21 wherein providing a sample of the rinsing solution comprises providing a discrete sample of the rinsing solution, wherein mixing the sample of the rinsing solution with a monitoring reagent comprises mixing the sample of the rinsing solution with a discrete sample of the monitoring reagent.

29. A system according to claim 28 wherein the discrete sample of the rinsing solution comprises approximately 0.2 milliliters and the discrete sample of the monitoring reagent comprises approximately 0.05 milliliters.

30. A system according to claim 28 wherein a volume ratio of the discrete sample of the rinsing solution to the discrete sample of the monitoring reagent is in the range of approximately 1:1 to approximately 5:1.

31. A system according to claim 28 wherein discrete samples of the rinsing solution and the monitoring reagent are mixed periodically.

32. A system according to claim 21 wherein the monitoring reagent comprises a chelating agent.

33. A system according to claim 32 wherein the chelating agent comprises at least one of an azo type organic compound, an eriochrome cyanine R type organic compound, chromeazurol-S(CAS), C₂₃H₁₃Cl₂Na₃O₉S, an 8-hydroxyquinoline derivative, 8-hydroxyquinoline, 5-sulfo-8-hydroxyquinoline, 5-chloro-7-iodo-8-hydroxyquinoline, 5,7-dichloro-8-hydroxyquinoline, 5.7-dibrome-8-hydroxyquinoline, tiron (1,2-dihydroxy-3,5-benzenedisulfonic acid disodium salt, monohydrate), hydroxy-2-(2-hydroxyphenylazo)benzene, lumogallion (5-chloro-2-hydroxy-3-(2,4-dihydroxyphenylazo)benzenesulfonic acid), and/or pyrocatechol violet (PV pyrocatechol sulfonphthalein).

34. A system according to claim 21 wherein the monitoring reagent comprises a chelating agent and a pH controller including at least one of a base and/or an acid.

35. A system according to claim 34 wherein the rinsing solution is alkaline and wherein the pH controller is acidic.

36. A system according to claim 34 wherein the rinsing solution is acidic and wherein the pH controller is alkaline.

37. A system according to claim 34 wherein the monitoring mixture has a pH level in the range of approximately 3 to 11.

38. A system according to claim 34 wherein the monitoring mixture has a pH level in the range of approximately 4.8 to 7.5.

39. A system according to claim 21 wherein the reagent comprises a chelating agent, water, and a pH controller including at least one of a base and/or an acid.

40. A method according to claim 21 wherein the rinsing solution comprises at least one of NH₄OH/H₂O₂/H₂O(SC1), H₂SO₄/H₂O₂, HCl/H₂O₂/H₂O, HNO₃/HF/H₂O, HF/H₂O₂, HF/NH₄F/H₂O, diluted HF, HF/NH₄F, HF/HNO₃/CH₃COOH, H₃PO₄, HNO₃/H₃PO₄/CH₃COOH, and/or de-ionized water.

41. A monitoring reagent for monitoring a level of a metal contamination in a rinsing solution, the monitoring reagent comprising:
a chelating agent that forms a complex compound with a metal when mixed with the rinsing solution including the metal; and
a pH controller comprising at least one of an acid and/or a base.

42. A monitoring reagent according to claim 41 wherein the chelating agent comprises at least one of an azo type organic compound, an eriochrome cyanine R type organic compound, chromeazurol-S(CAS), C₂₃H₁₃Cl₂Na₃O₉S, an 8-hydroxyquinoline derivative, 8-hydroxyquinoline, 5-sulfo-8-hydroxyquinoline, 5-chloro-7-iodo-8-hydroxyquinoline, 5,7-dichloro-8-hydroxyquinoline, 5.7-dibrome-8-hydroxyquinoline, tiron (1,2-dihydroxy-3,5-benzenedisulfonic acid disodium salt, monohydrate), hydroxy-2-(2-hydroxyphenylazo)benzene, lumogallion (5-chloro-2-hydroxy-3-(2,4-dihydroxyphenylazo)benzenesulfonic acid), and/or pyrocatechol violet (PV pyrocatechol sulfonphthalein).

43. A monitoring reagent according to claim 41 wherein the chelating agent comprises at least two of an azo type organic compound, an eriochrome cyanine R type organic compound, chromeazurol-S(CAS), C₂₃H₁₃Cl₂Na₃O₉S, an 8-hydroxyquinoline derivative, 8-hydroxyquinoline, 5-sulfo-8-hydroxyquinoline, 5-chloro-7-iodo-8-hydroxyquinoline, 5,7-dichloro-8-hydroxyquinoline, 5.7-dibrome-8-hydroxyquinoline, tiron (1,2-dihydroxy-3,5-benzenedisulfonic acid disodium salt, monohydrate), hydroxy-2-(2-hydroxyphenylazo)benzene, lumogallion (5-chloro-2-hydroxy-3-(2,4-dihydroxyphenylazo)benzenesulfonic acid), and/or pyrocatechol violet (PV pyrocatechol sulfonphthalein).

44. A monitoring reagent according to claim 41 wherein the chelating agent comprises an azo type organic compound and at least one of an eriochrome cyanine R type organic compound, chromeazurol-S(CAS), C₂₃H₁₃Cl₂Na₃O₉S, an 8-hydroxyquinoline derivative, 8-hydroxyquinoline, 5-sulfo-8-hydroxyquinoline, 5-chloro-7-iodo-8-hydroxyquinoline, 5,7-dichloro-8-hydroxyquinoline, 5.7-dibrome-8-hydroxyquinoline, tiron (1,2-dihydroxy-3,5-benzenedisulfonic acid disodium salt, monohydrate), hydroxy-2-(2-hydroxyphenylazo)benzene, lumogallion (5-chloro-2-hydroxy-3-(2,4-dihydroxyphenylazo)benzenesulfonic acid), and/or pyrocatechol violet (PV pyrocatechol sulfonphthalein).

45. A monitoring reagent according to claim 41 wherein the rinsing solution is alkaline and wherein the pH controller is acidic.

46. A monitoring reagent according to claim 45 wherein the monitoring reagent has a pH level in the range of approximately 1 to 3.

47. A monitoring regent according to claim 45 wherein the pH controller comprises acetic acid.

48. A monitoring reagent according to claim 41 wherein the rinsing solution is acidic and wherein the pH controller is alkaline.

49. A monitoring reagent according to claim 48 wherein the monitoring reagent has a pH level in the range of approximately 8 to 10.

50. A monitoring reagent according to claim 48 wherein the pH controller comprises ammonia.

51. A monitoring reagent according to claim 41 further comprising water.

52. A monitoring reagent according to claim 51 wherein the chelating agent comprises in the range of approximately 0.0001 to approximately 0.1 weight percent of the monitoring reagent.

53. A monitoring reagent according to claim 51 wherein the pH controller comprises in the range of approximately 1 to approximately 30 weight percent of the monitoring reagent.
